(19) United States
(12) Patent Application Publication
(10) Pub. No.: US 2024/0149008 A1
(43) Pub. Date: May 9, 2024

(54) COGNITIVE THERAPY USING VIRTUAL ENVIRONMENTS

(71) Applicant: Daniel Augustine ROBINSON, Marina Del Rey, CA (US)

(72) Inventor: Daniel Augustine ROBINSON, Marina Del Rey, CA (US)

(21) Appl. No.: 18/281,475

(22) PCT Filed: Mar. 14, 2022
(86) PCT No.: PCT/US2022/020172

§ 371 Date: Sep. 11, 2023

Related U.S. Application Data
(60) Provisional application No. 63/160,345, filed on Mar. 12, 2021.

(51) Int. Cl. A61M 21/00; G09B 19/00; G16H 10/60

(52) U.S. Cl. CPC A61M 21/00; G09B 19/00; G16H 10/60; A61M 2021/005; A61M 2021/0077; A61M 2205/507

(57) ABSTRACT

A method for treating a person suffering from a negative psychologic reaction when a past traumatic event is recalled, the method comprising: (a) accessing clinical data of a person's mental state relating to the event causing the negative psychologic reaction; (b) creating a mixed reality (XR) environment for the person, the XR environment comprising at least a display; and

(c) presenting sequential content on the display over a period of time, wherein the sequential content comprises multiple depictions of the event at increasing levels of immersion, including an initial depiction of the event at an initial immersion level, and a final depiction of the event at a final immersion level, wherein the final immersion level is the highest immersion level in the XR environment.

### REFERENCE TO RELATED APPLICATION

This application is based on U.S. Provisional Application No. 63/160,345, filed Mar. 12, 2021, the entire disclosures of which are incorporated herein by reference.

### TECHNICAL FIELD

The present disclosure relates generally to immersion systems and methods as clinical aids used in cognitive therapy.

### BACKGROUND

Cognitive therapy is a highly specialized field that requires expert clinicians to evaluate and treat people with a variety of disorders and diseases. Researchers are continually looking for better and more effective treatments. Recently, there has been considerable research around the idea that the use of psychedelic drugs during cognitive therapy may be one new effective tool. The general concept is that the drug can open new pathways in the brain and allow a clinician to obtain better access to a person's condition. There remains a need for further development of therapies and tools for clinical use in cognitive therapy sessions.

### SUMMARY

Embodiments of the present disclosure involve a computer software product, systems, and methods to assist a person during psychologic counseling. The systems may involve creating a virtual environment with controllable levels of immersion to assist in trauma therapy or addiction therapy.

### BRIEF DESCRIPTION OF THE FIGURES

The details of particular implementations are set forth in the accompanying drawings and description below. Like reference numerals may refer to like elements throughout the specification. Other features will be apparent from the following description, including the drawings and claims. The drawings, though, are for the purposes of illustration and description only and are not intended as a definition of the limits of the disclosure.

FIG. 1 illustrates an XR system for use in cognitive therapy in accordance with principles of the present invention.

FIG. 2 illustrates variable immersion levels in accordance with the principles of the present invention.

### DETAILED DESCRIPTION

The inventor has discovered that the use of mixed reality (XR) (which includes augmented reality, (AR), and virtual reality (VR)), associated computer systems, software, and user interfaces in clinical sessions of cognitive therapy can be useful and effective for the treatment of a person's disability, disorder, or disease. The use of XR can create an immersive experience for a patient that is otherwise not possible. A clinician and/or the patient may also control the virtual environment such that the experience is properly controlled. The inventor also discovered that the combined use of psychedelic drugs and XR can open new therapeutic pathways and create effective treatment platforms.

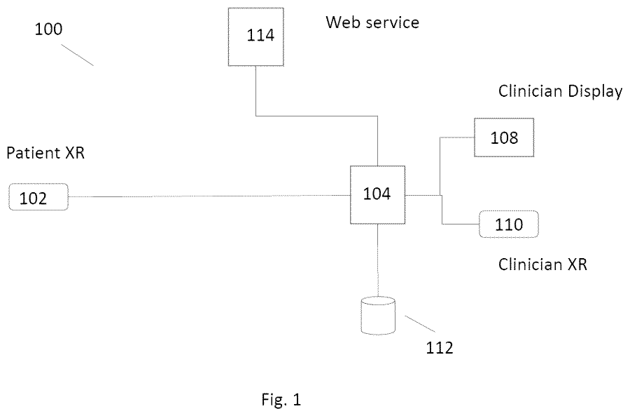

FIG. 1 illustrates an XR system 100 for use in cognitive therapy in accordance with principles of the present invention. The patient may wear an XR head-mounted device 102 through which he can experience a virtual environment. The XR head-mounted device may be part of a larger system used to provide the patient with a proper immersion experience. The larger system may include an audio system, haptic system, movable table, stand, or chair to cause the patient to move in coordination with the virtual experience.

The XR system 100 may include a computer system 104 with a clinician computer screen 108 and clinician XR head-mounted device 110, a local database 112, a remote computer service and database 114, and other systems or services to assist a clinician with the application of the therapeutic immersion experience. For example, a clinician may use the computer system 104 to recall patient diagnosis information, reference experience information describing past patient events, traumas, life experiences, etc. The patient diagnosis information may help the clinician in selecting XR experience data (e.g., synthetically developed content, photographic images, videos) describing events or experiences, for presentation to the patient. The XR experience data may be stored locally in the local database 112, in the remote database 114, etc. The computer system 104 and/or the remote computer service and database 114 may generate visual, audio, haptic, etc. content to be presented to the patient in the XR system 100.

The XR system 100 may also have a clinician experience system, including a XR head-mounted device 110, computer screen 108, etc. to view the virtual environment being provided to the patient. The clinician may use the clinician experience system to watch, listen, feel, etc. the experience as presented to the patient. The patient may also be automatically observed (e.g. eye movements, facial expressions, pulse, blood pressure, sweat, breathing rate, breath depth) to track the patient's reactions during the clinically presented immersive experience. The patient observations may be presented to the clinician in the clinician experience system. For example, feedback may be presented on the clinician's computer screen or through the clinician's XR head-mounted device. The clinician may then adjust (e.g., increasing an immersion level, decreasing an immersion level, stopping the virtual experience) based on the observation data. The clinician may have a user interface where he can control the level of immersion or other aspects of the virtual environment presented to the patient.

In embodiments, the XR system 100 may be used in a clinical setting under the direction and control of a clinician. The immersive experience the patient has may itself open pathways in the patients conscious or subconscious that are otherwise difficult or not practically possible to open. To assist in opening pathways (e.g., in addition to those pathways opened through the immersive experience, or to increase the access to or reception by a new pathway) a psychedelic drug(s) may be administered to the patient. The XR system 100 may be used to gain an understanding of the patient's level of affect by the drug or XR experience. The XR system 100 may also be used to understand when the patient's level of affect is right for administration of the virtual experience. The patient's affect may not be appropriate for minutes, hours, day(s), etc.

The XR system 100 may be used for a wide variety of cognitive therapies and various psychedelic drugs, or combination of drugs, may be used based on the therapeutic need of the patient. For example, a patient may be suffering from a past trauma (e.g., PTSD, an abusive environment, combat environment, accident, personal loss) and an immersive experience may be generated, tailored, or selected to be presented through the XR system 100. One type of cognitive therapy used in past trauma situations is referred to a desensitization. In psychology, desensitization involves a treatment or process that diminishes a person's emotional responsiveness to a negative, aversive or positive stimulus after repeated exposure to it. Desensitization also occurs when an emotional response is repeatedly recalled in situations in which the person's reaction tendency that is associated with the emotion proves irrelevant or unnecessary. The person can be taught to understand that the traumatic experience does not have to evoke a bad, sad, anxious, hostile, or other reaction by associating the traumatic experience with a learned response.

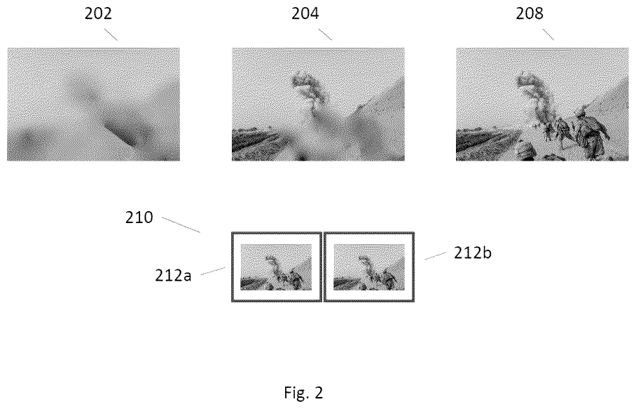

There are many ways to increase the patient's level of immersion and engagement in the XR system (e.g., changing imagery clarity, resolution, focus, color pallet, etc. and/or changing the playback speed of a video, present an image in 3D, present a video in 3D, etc. and/or changing the quality and intensity of audio and haptic effects, etc.). FIG. 2 illustrates varying degrees of visual content immersion that may be used in the XR system 100. A patient may be presented with lower quality or blurred content 202 early in therapy or dependent on the patient's stage of recovery or sensitivity to such a synthetic environment. The patient may, at a later point in his recovery, be presented with more detailed imagery 204 and then move into very clear and detailed imagery 208. The progression of visual immersion may continue by presenting imagery and video (e.g., computer generated, actual scene footage, etc.) in three dimensions (3D). One such 3D technology that may be used is an augmented reality, virtual reality, mixed reality or other system where the visual content is presented from slightly different perspectives to each of the patient's eyes through independently controlled displays 212a and 212b or display segments. The different perspectives may replicate a person's natural 3D experience due to the person's eye separation. This may be used when the visual experience is close up, which is generally referred to as divergence. The colorization of the content presented to each eye may be different to create a desired effect.

In embodiments, an XR system 100 may include an adjustable transmission display that can be controlled to go from a substantially transparent, see-through, mode and a non-transmissive mode. This adjustable display may be controlled to gradually decrease or increase its transmissivity or change to a preset transmissivity. There may be more than one present transmissivity. The level of transmissivity may be decreased to increase the level immersion for the patient. For example, a head mounted display may have an electrochromic surface in the line of sight of the display and the electrochromic surface may be controlled to vary the level of transmissivity. As another example, replaceable lenses of varying degrees of transmissivity may be snapped onto a head mounted display to change the transmissivity of the display from the user's perspective.

Another desensitization therapy that may be presented in the XR system 100 is eye movement desensitization and reprocessing (EMDR). Content may be presented to the patient that moves and the patient is instructed to follow the movement. This could be done in connection with a psychedelic affect or not. The XR system 100 may include eye movement tracking systems (e.g. cameras in the frame of glasses facing the person's eyes) such that the clinician may track the patients performance. The clinician may be remote from the patient and the eye tracking system may provide feedback to help ensure the patient is doing it correctly. Remote EMDR, or other XR therapies, is a therapy that may benefit from a controlled and monitored experience through a XR system 100.

The movements, in EMDR may be rapid, rhythmic, etc. and may be presented at the same time, or at a separate time, from content that is presented to help the patient recall the trauma suffered. For example, content of varying degrees of immersion may be presented and then followed by EMDR presented content. The content of varying degrees of immersion may be presented at the same time as the EMDR content. One may overlay the other. For example, blurry content may be presented and EMDR content may be presented over it. The EMDR content may present the clinician with another tool in the XR environment to be used at various times, which may be under the control of the clinician. For example, the clinician may feel that the patient is in the right condition and then start an EMDR session.

A patient may have an addiction disorder or disease and the XR system 100 may be used in therapy to help the person control or overcome his addiction. There are instances when a person does not know how to cope with certain situations and their lack of coping skills causes them to use drugs, alcohol, etc. just to cope, which can lead into addiction. There are also times when a person starts using, or abusing, drugs or alcohol at a young age when they would normally, that is without the drugs of alcohol, would be learning to cope with different situations. This can lead to a situation where the person never learns how to cope and continues to use as his coping mechanism.

Addiction therapy may have several stages of recovery. For example, the person may have to deal with his physical, chemical, hormonal, etc. changes that have been changed by using. It may take a person six to twelve months after getting sober to get their body and mind to a condition where they can begin to explore and change the underlying issues leading to addiction. During this period the person may suffer from irritability, depression, acute issues, etc. and the person needs to learn how to cope with these feelings and fight off a relapse. During this period, a clinician may use the XR system 100 to provide the patient with a safe immersion environment tailored to teach the person what is happening to his chemistry, hormones, etc. and to teach methods to cope during this period.

Once a person with an addition problem stabilizes after detox and the effects of the use on his body and mind, a clinician may use the XR system 100 to provide the patient with content causing him to recall his early uses, why he used, etc. and also teach how he did not learn to cope in this or later periods.

A clinician may use the XR system 100 while using cognitive triangle management techniques. The cognitive triangle describes how negative thoughts can generate negative feeling which can cause negative behaviors. the negative behaviors reinforce the negative thoughts and the triangle of negativity continues. A clinician may use the XR system 100 to present an immersive experience to break the negative cycle. The content, for example, may cause the patient to recall negative thoughts so the clinician can talk about them with the patient. Other positive content may also be presented as a way of combating the negative cycle. For example, the negative thoughts may relate to a social anxiety so the clinician may present a social environment to cause the patient to experience that anxiety. In this period the clinician may be able to observe the patient in the condition without causing harm by bringing the person into an uncontrolled environment.

Once the patient is experiencing the anxiety, the clinician may use the XR system 100 to alter the immersive experience to demonstrate how the patient may cope better in such situations. The content may show the patient that bad things are not going to happen in such situations by having the patient virtually walk through the virtual environment while using a management technique.

In embodiments, a patient's brain activity may be monitored (e.g. with a wearable sensor(s), remote sensor, imaging, electric sensors) before, during, or after therapy with the XR system 100. This may provide the clinician with feedback relating to the patient's response to the therapy, whether during or following a therapy session.

In embodiments, a brain-computer interface may be used to facilitate monitoring of the patient's brain and/or to facilitate the person to use his brain during a therapy session. For example, a clinician, or the XR system, may prompt a patient to control a device with his mind, through a brain-computer interface, to cause the person to focus on that task. This focus technique may be used in conjunction with varying degrees of immersion or other technologies described herein to further the recovery of the patient.

While some therapy examples have been provided herein, they are provided as non-limiting examples of how an XR system 100 may be used to treat a patient and other therapy techniques may use such principles as described herein.

The terms “augmented reality,” “AR,” “virtual reality,” “VR,” and “mixed reality” “XR” as used in this disclosure generally refer to computer systems that include a visual presentation system to create an immersive visual experience (e.g. a head mounted computer display, holographic projection within an environment). In embodiments, augmented reality or AR may refer to a system that that provides a user with a see-through computer display in which digital content is provided. In embodiments, virtual reality or VR may refer to a system that provides a user with a non-see-through computer display that covers substantially all of the user's vision. In embodiments, mixed reality or XR may refer to a system that provides either or both a see-through and non-see-through computer display; the computer display may be switchable to change between transmissive, or a level of transmissivity, and substantially non-transmissive. These terms may be used interchangeably within this patent application unless specificity is drawn to a particular style.

The term “psychedelic drug” or “psychedelic” as used in this disclosure generally refers to a drug that when taken by a person create a non-ordinary state of consciousness in the person. Such a drug may cause specific psychological, visual, and auditory changes and/or a substantially altered state of consciousness. Such a drug may cause hallucinations or be referred to as a hallucinogenic. Some examples of a psychedelic include psychedelic mushrooms, mescaline, acid, Lysergic acid diethylamide (LSD), psilocybin and Dimethyltryptamine (DMT).

Each of the processes, methods, and algorithms described in the preceding sections may be embodied in, and fully or partially automated by, code modules executed by one or more computers or computer processors. The code modules may be stored on any type of non-transitory computer-readable medium or computer storage device, such as hard drives, solid state memory, optical disc, and/or the like. The processes and algorithms may be implemented partially or wholly in application-specific circuitry. The results of the disclosed processes and process steps may be stored, persistently or otherwise, in any type of non-transitory computer storage, such as, e.g., volatile or non-volatile storage.

The various features and processes described above may be used independently of one another, or may be combined in various ways. All possible combinations and sub-combinations are intended to fall within the scope of this disclosure. In addition, certain methods or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It will also be appreciated that various items are illustrated as being stored in memory or on storage while being used, and that these items or portions thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (“ASICs”), standard integrated circuits, controllers (e.g., by executing appropriate instructions, and including microcontrollers and/or embedded controllers), field-programmable gate arrays (“FPGAs”), complex programmable logic devices (“CPLDs”), etc. Some or all of the modules, systems, and data structures may also be stored (e.g., as software instructions or structured data) on a computer-readable medium, such as a hard disk, a memory, a network, or a portable media article to be read by an appropriate device or via an appropriate connection. The systems, modules, and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, the present invention may be practiced with other computer system configurations.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method for treating a person suffering from a negative psychologic reaction when a past traumatic event is recalled, the method comprising:
accessing clinical data of a person's mental state relating to the event causing the negative psychologic reaction;
generating a mixed reality (XR) environment for the person, the XR environment comprising at least a display; and
presenting content on the display over a period of time, wherein the content comprises multiple depictions of the event at increasing levels of visual clarity, including an initial depiction of the event at a low resolution level, and a final depiction of the event at a higher resolution level.

2. The method of claim 1, wherein the person is exposed to the XR environment at a time when the person is affected by a psychedelic drug and recording the person's response.

3. The method of claim 1, wherein the clinical data was obtained at a time when the person was affected by a psychedelic drug.

4. The method of claim 1, wherein the negative psychologic reaction relates to post traumatic stress syndrome.

5. The method of claim 1, wherein the multiple depictions are three-dimensional representations of the event.

6. The method of claim 1, wherein the period is from one week to one year.

7. (canceled)

8. A system for treating a person suffering from a negative psychologic reaction when a past traumatic event is recalled, the system comprising:
a database;
a display; and
a computer configured for
accessing the database to obtain clinical data of a person's mental state relating to the event causing the negative psychologic reaction;
creating a mixed reality (XR) environment for the person using at least the display; and
presenting content on the display over a period of time, wherein the content comprises multiple depictions of the event at increasing levels of visual clarity, including an initial depiction of the event at an initial low resolution level, and a final depiction of the event at a final higher level.

9. The system of claim 8, wherein the display is worn by the person.

10. The system of claim 8, wherein the multiple depictions are three-dimensional representations of the event.

11. The system of claim 8, wherein the period is from one week to one year.

12. (canceled)
